(19) United States
(12) Patent Application Publication
(10) Pub. No.: US 2010/0022927 A1
(43) Pub. Date: Jan. 28, 2010

(54) BODY LIMB CAST WITH KEY ADJUSTABLE OUTER CAGE AND INNER FOAM SUPPORT MEMBERS FOR MAINTAINING BREATHE-ABILITY

(75) Inventor: Miguel A. Linares, BLOOMFIELD HILLS, MI (US)

Correspondence Address: GIFFORD, KRASS, SPRINKLE,ANDERSON & CITKOWSKI, P.C, PO BOX 7021, TROY, MI, 48007-7021, US

(73) Assignee: LINARES MEDICAL DEVICES, LLC, Auburn Hills, MI (US)

(21) Appl. No.: 12/504,721

(22) Filed: Jul. 17, 2009

Related U.S. Application Data
(60) Provisional application No. 61/083,035, filed on Jul. 23, 2008.

(51) Int. Cl. A61F 5/00

(52) U.S. Cl. 602/5

(57) ABSTRACT

A body limb cast including an elbow or knee joint supporting element having a first perimeter edge and a second opposite perimeter edge. A first splint subassembly is secured in engaging and extending fashion from the first perimeter edge, a second splint subassembly secured in engaging and extending fashion from the second perimeter edge of the joint supporting element. Cushioning elements extend from an inside of each splint subassembly and support the surfaces of a patient's limb upon assembly of the joint supporting element and splints. The splint subassemblies each further include a plurality of individual, lengthwise extending and assembleable members extending around a perimeter about the patient's limb.

### CROSS-REFERENCE TO RELATED APPLICATIONS

This Application claims the benefit of U.S. Provisional Application 61/083,035 filed on Jul. 23, 2008.

### FIELD OF THE INVENTION

The present invention relates generally to a wearable cast for installation upon a patient's arm or leg. More specifically, the present invention discloses an adjustable cast having a joint situated support element, from which extends at least one surrounding cage for assembling over a patient's damaged limb and including key access diameter adjustment clamps. A foamy/sponge-like material is interposed between an interior facing surface of the cage and the patient's limb and, in combination with the adjustments made to the cast, both cushions and biases the patient's limb in an immobilizing and comfort inducing fashion.

### BACKGROUND OF THE INVENTION

The prior art is documented with examples of splint and cast assemblies, the purpose for which being to support an injured limb such as an arm or a leg of a wearer. One example of a universal splint for use in immobilizing a body extremity is described in U.S. Pat. No. 4,280,490, issued to Santy, and which disclose a backboard with strap fasteners for securing across a person's back. A double hinge upper arm and lower forearm arrangement extends from the backboard and are individually lockable to restrain the injured extremity.

Sterling, U.S. Pat. No. 4,019,504, teaches a medical splint kit with reusable components used during X-raying of a body part. One or more splint members are formed from a X-irradiation material such as an acrylic plastic and the splint also includes sections of loops or hooks to facilitate affixation to the body.

Additional examples of braces for use with articulated limbs include such as the brace in Bledsoe, U.S. Pat. No. 4,407,276, the knee immobilizier of Grim, U.S. Pat. No. 7,513,881 and the universal leg brace system of Hutson, U.S. Pat. No. 4,494,534.

### SUMMARY OF THE INVENTION

The present invention discloses an assembleable and adjustable body limb cast for installation over such as an arm or leg. An elbow or knee joint supporting element is provided exhibiting first and second assembleable halves jointly defining an angled profile having a first perimeter edge and a second opposite perimeter edge. A first splint subassembly is secured in engaging and extending fashion from the first perimeter edge, a second splint subassembly secured in engaging and extending fashion from the second perimeter edge of the joint supporting element. Cushioning elements extend from an inside of each splint subassembly and support the surfaces of a patient's limb upon assembly of the joint supporting element and splints.

The splint subassemblies each further include a plurality of individual, lengthwise extending and assembleable members extending around a perimeter about the patient's limb. Additional features include comprising at least one diameter adjusting clamp associated with each of the first and second splint subassemblies. A band encircles at least one location associated with the splint subassemblies, a diameter of which is adjusted by the adjusting clamp.

The elbow joint further includes at least one diameter adjusting clamp, with the joint supporting elements and splint subassemblies each further preferably exhibiting a durable and polymeric material. Other features include the perimeter extending edges of the joint supporting elements having a rail support surface upon which opposing locations of the splint subassemblies are resistively engaged. A key access feature can be associated with the diameter adjusting clamp, such as to limit access to authorized medical personnel.

### BRIEF DESCRIPTION OF THE DRAWINGS

Reference will now be made to the attached drawings, when read in combination with the following detailed description, wherein like reference numerals refer to like parts throughout the several views, and in which.

FIG. 1 is a perspective view of an adjustable cage splint according to a first preferred embodiment of the present invention and for application to a patient's arm;

FIG. 2 is an enlarged and slightly rotated perspective illustration of the cage shown in FIG. 1;

FIG. 3 is a further enlarged view of a lower cage section and better illustrating the application of a special tool for adjustable tightening and retightening of each clamp;

FIG. 4 is a further enlarged view of the cage diameter adjustment provided by the clamp tightening screw;

FIG. 5 is a side view of the adjustable cage splint and better showing the features associated with the interconnecting middle joint support, from which extend lower and upper arm extending cage sections;

FIG. 6 is an exploded view of an adjustable cage splint according to a second preferred embodiment for application about a patient's leg;

FIG. 7 is an enlargement of the exploded view of FIG. 6 and better showing the feature of pluralities of assembleable calf and thigh splints engageable with the central, two piece assembleable knee portion;

FIG. 8 is an upper assembled perspective view of the leg cast and further illustrating the feature of the tie tightening/adjustment screws and the manner in which the upper and lower splints align and lock with opposite perimeter extending edges associated with the central assembleable knee portion;

FIG. 9 is a top view of the leg cast of FIG. 6 and better illustrating the arrangement and configuration of the inner foam supporting portions;

FIG. 10 is a front plan view of the assembled leg cast; and

FIG. 11 is a rotated and rearward looking perspective view of the leg cast also shown in FIG. 10.

### DETAILED DESCRIPTION OF THE PREFERRED EMBODIMENTS

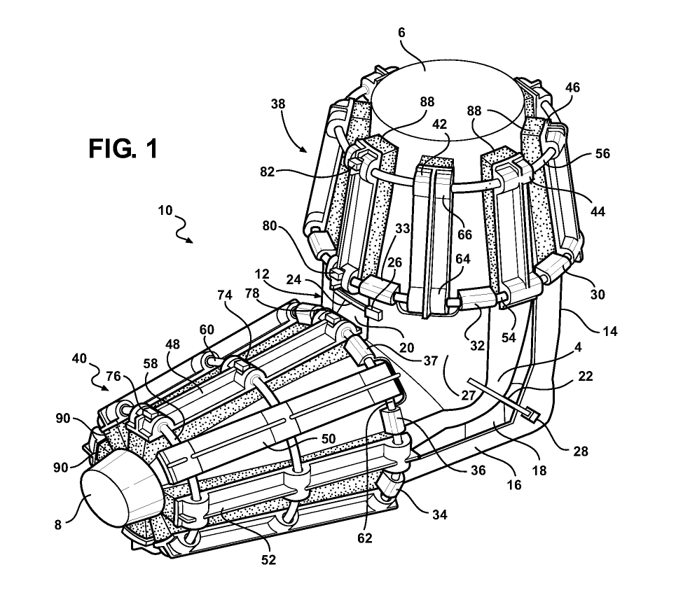

Referring now to FIG. 1, a perspective view is shown at 10 of an adjustable cage splint according to a first preferred embodiment of the present invention for application to a patient's arm. As previously described, the present inventions disclose an adjustable cast including a joint situated support element, and to which is secured in extending fashion at least one surrounding cage for assembling over a patient's damaged limb, the cage further including key access diameter adjustment clamps. A foamy/sponge-like material is interposed between an interior facing surface of the cage and the patient's limb and, in combination with the adjustments made to the cast, provides both cushioning and biasing of the patient's limb in an immobilizing and comfort inducing fashion.

Illustrated generally at 12 is an intermediately positioned and joint support element, this typically being constructed of a durable polymeric or possibly lightweight metallic construction. As with the cage subassemblies to be subsequently described, the joint support element 12 is constructed of a multi-piece assembleable (or hinged) construction and which is designed to encase, in assembled and predetermined spaced fashion, a joint area proximately located to a patient's injured limb. The joint support element 12 as further shown includes first 14 and second 16 sides arrayed at a desired angular orientation (and such as in the illustrated embodiment is intended to position the user's upper arm and forearm in a like arrangement), it further being understood that the joint support element 12 can also be reconfigured to reproduced in other configurations.

The multi-piece joint support element 12 is further evidenced by a series of length extending apertures, or individual spacer channels, see at 18, 20 et seq., these providing the dual features of breath-ability as well as diameter adjustability in response to the particular sizing needs of a given cast to a particular individual. Along these lines, it is envisioned that a subset number of cast assemblies (these including joint support element and attachable cage subassemblies) can be provided, such as small, medium and large, and which can apply to a wide range of patients.

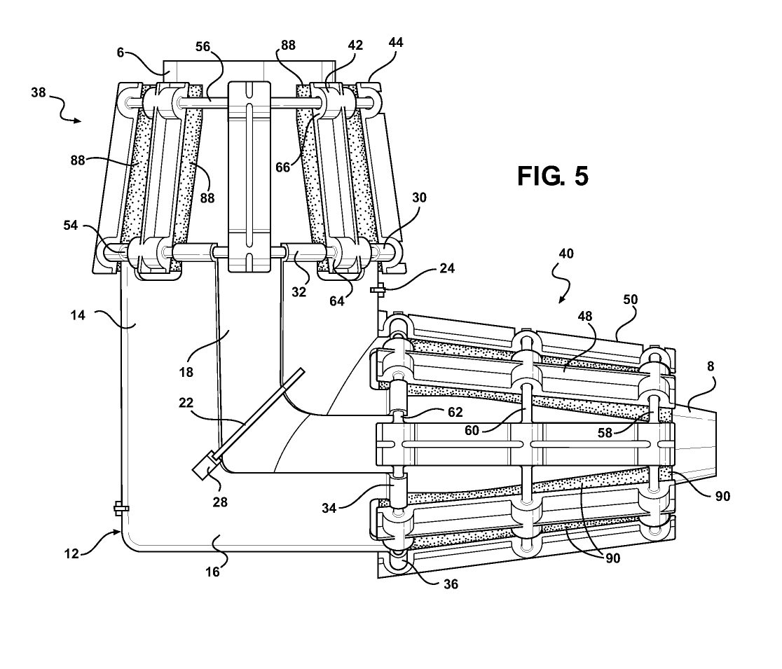

As shown in each of illustrations associated with FIGS. 1-6 the joint support element 12 is pre-positioned (such as assembling individual pieces or otherwise closing its halves in a clam-shell like fashion) about the user's elbow joint, shown at 4 in FIG. 1, and which is defined between an upper arm 6 and a lower arm 8. Additional features associated with the joint support element 12 include a plurality of joint (elbow) tightening clamps, see as 22 and 24, these extending widthwise between spaced apart sections of the joint support element 12 and bridging the intervening spacer channels 18, 20, et. seq.

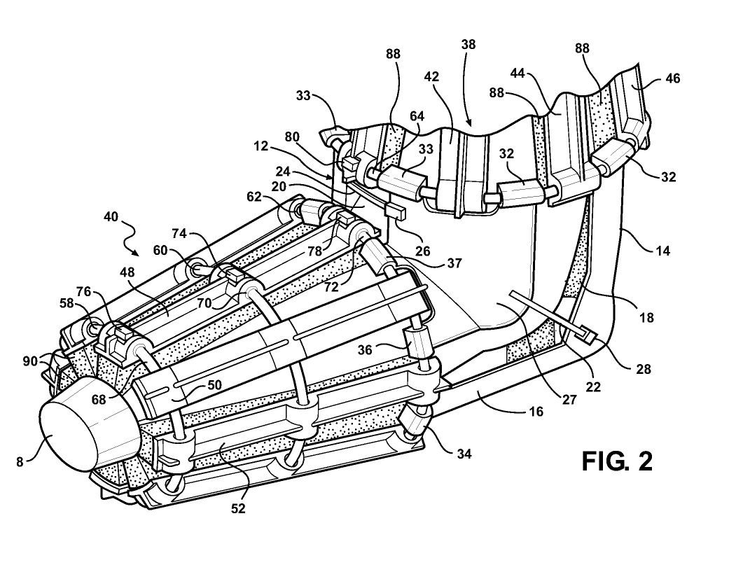

As also shown in the enlarged illustration FIG. 2, selected strip portion 24 associated with a tightening clamp is fixed to a first crosswise extending surface of the joint support element 12, the clamp extending widthwise across open channel 20 and engaging through a receiver 26 formed upon a surface of a succeeding and spaced apart piece, e.g. at 27 in FIGS. 1 and 2, of the assembleable joint support element 12. The adjustable clamps shown, including both the front extending clamp 24 and angled side extending clamps 22 (a second of which is hidden from view in FIGS. 1 and 2), can each be adjustable by translating the elongated strip portion relative to associated locking structure incorporated within an associated interior of each receiver (e.g. again at 26 and as further shown at 28 in reference to each of clamps 24 and 22). Although not clearly shown, the strip portions associated with each of the clamp undersides can also be configured with a serrated profile, such as in one non-limiting application and which may engage with a like mating profile in the receiver interior, this assisting in properly sizing and immobilizing the assembled joint support elements 12 upon such as the patient's elbow 4.

The individual and chancel spaced-apart surfaces of the first 14 and second 16 extending sides of the joint support element 12 each further exhibit individual, arcuate extending and interior aperture defined portions, see at 30, 32, 33 et. seq. associated with upper arm extending side 14 and further at 34, 36, 37, et. seq., associated with lower extending side 16. The individual pluralities of the spaced apart and arcuate edge extending portions establish perimeter defining locations associated with each of upper and lower arm extending edges of the joint support element 12.

With her reference now to the upper arm and lower arm cage (or splint) subassemblies, generally referenced at 38 and 40, respectively, each is capable of being assembled in engaging fashion with the arcuate perimeter defining portions 30, 32, 33 of side 14 and 34, 36, 37 of side 16 of the multi-piece joint support element 12, and so that either or both the patient's upper and lower arms are properly supported and immobilized. The upper arm cage subassembly 38 includes a plurality of lengthwise extending and spaced apart members 42, 44, 46, et. seq., whereas the lower arm cage subassembly 40 includes a further similar plurality of members 48, 50, 52, et. seq.

The individual plurality of lengthwise extending, spaced apart and perimeter defining portions of each cage subassembly also each finer include perimeter encircling support rings, see as shown at 54 and 56 for upper arm cage subassembly 38 and further at 58, 60 and 62 for lower arm cage subassembly 40. Each of the lengthwise spaced apart members further include a crosswise extending aperture (or fixed engaging location) defined through a reinforced location of a given member, at end and (in the instance of the lower members) intermediate locations, each for receiving a given perimeter defining ring.

For purposes of each of description, selected upper arm lengthwise member 42 includes end defined crosswise and arcuate shaped aperture defining portions 64 and 66, these associated with circumferential engaging and supporting rings 54 and 56. Selected lower arm lengthwise member 48 includes crosswise and arcuate shaped aperture defined portions 68, 70 and 72 associated with rings 58, 60 and 62. As will be explained in reference to the clamp adjustment assemblies, the rings 54 and 56 (upper cage) and 58, 60 and 62 (lower cage) can be fixed or slidably disposed with respect to some or all of each of the individual and lengthwise extending sections associated with the upper 38 and lower 40 cage assemblies.

As further shown, the joint element proximate rings including those at 62 (lower arm) and 54 (upper arm) engage, such as in a snap-fit fashion, within the individual, arcuate extending portions, see again at 30, 32, 33 et. seq. associated with upper arm extending side 14 and further at 34, 36, 37 et. seq., associated with lower extending side 16. In this fashion, and upon first assembling the upper and lower cage subassemblies 38 and 40 about the patient's upper and lower arm (such as by individually assembling each of the individual pluralities of lengthwise defined members 42-46 and 48-52), the selected rings 54 and 62 are snap fit into place with the individual, arcuate extending portions, once again at 30, 32, et. seq. associated with upper arm extending side 14 of the joint supporting element 12, as well as further at 34, 36, et. seq., associated with lower extending side 16.

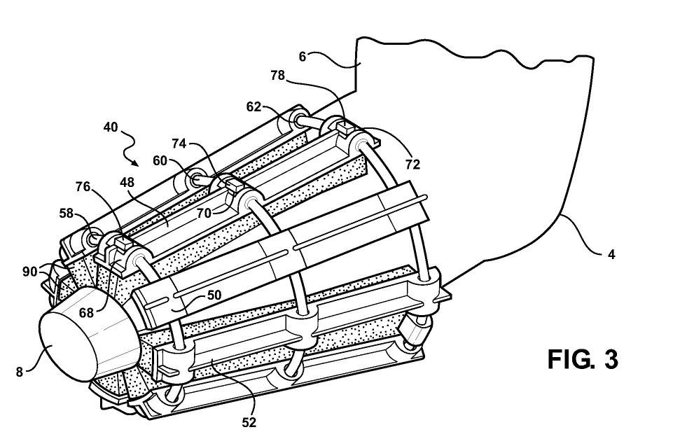

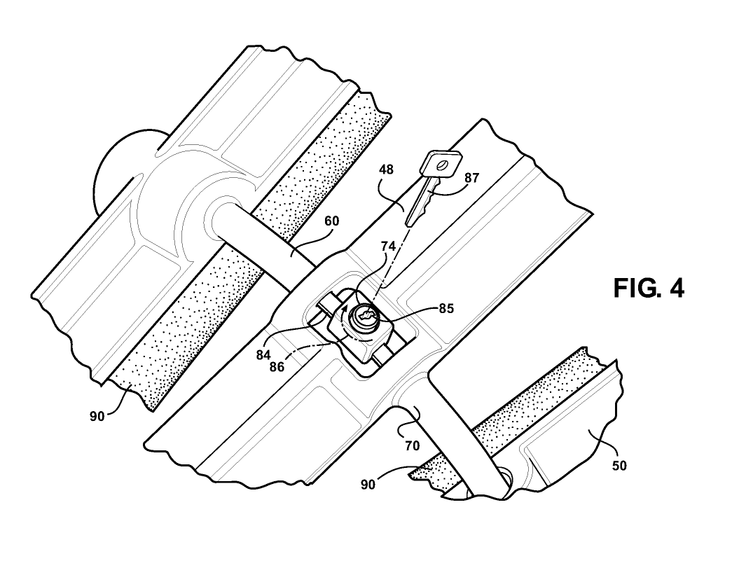

As best shown in each of FIGS. 3 and 4, diameter adjustment tightening of each cage subassembly is further accomplished through the provision of a tightening tool (not shown) which is employed to actuate each of a series of clamps associated with both the lower and upper cage subassemblies. The lower cage subassembly includes a central clamp 74 (as well as first and second end defined clamps 76 and 78 as further shown in FIGS. 1 and 2 and which are also associated with the selected lengthwise extending and spaced apart member 48), the clamps 74-78 being arranged at linear spaced apart locations associated with the lengthwise member 48 and through which correspondingly extend the supporting and securing rings 58, 60 and 62. The upper cage subassembly likewise includes a pair of clamps 80 and 82 (see again FIG. 1) likewise arranged proximate the upper aim defined rings 54 and 56.

As best shown again in FIGS. 3 and 4, selected tightening clamp 74 includes an interior and perimeter extending band or strip (see at 84 in FIG. 4), this exhibiting a diameter less than the associated ring 60 and extending inside a hollow interior defined in the ring 60. In this arrangement, the rings are integrally formed with some or all of the individual and lengthwise extending members (e.g. 48, 50 and 52 as again referencing lower cage subassembly 40) and, upon assembling such members such as in first and second clamshell sections or the like so that the perimeter defined and translating strips 84 communicate all the way around each ring interior, the clamps are tightened (such as by employing a tip of a given tool in a rotating fashion as shown at 86 in FIG. 4). In this fashion, each band 84 is drawn together or otherwise pinched utilizing an appropriate inter-engaging mechanism incorporated into the tightening clamp 74 in order to compress the individual, generally parallel and lengthwise extending members 48, 50 and 52 (as well as at 42, 44, and 46 for upper cage subassembly).

The clamps may each include key-access for permitting such as only an authorized medical professional from accessing and/or readjusting each cage subassembly (see key-slot 85 defined in illustrated clamp 74 which receives surgeons key 87). The material content and or construction of the cage subassemblies is further such that spaced apart and lengthwise extending members are capable of being displaced to some incremental degree in widthwise extending and adjustable fashion. This can also be accomplished by a number of ways, including incising a portion of each ring so that a range of diameter adjustment is allowable upon accessing each clamp. Also, and as previously indicated, selected lengthwise members can be slidably engaged relative to the encircling rings and, in combination with other fixed locations established between the lengthwise members and rings, will allow a selected degree of diameter defining adjustment.

As illustrated in each of FIGS. 1-5, foam cushioning elements are also provided in individual pluralities and are secured in inwardly displacing and biasing fashion between each of the individual pluralities of lengthwise extending supports associated with both the upper 38 and lower 40 arm cage subassemblies. A first plurality of such cushioning elements are shown at 88 and are biasingly sandwiched between the upper cage members 42, 44, 46 et, seq., and the patient's upper arm 6, whereas a further plurality of cushioning elements 90 are likewise sandwiched between the lower cage members 48, 50 and 52, et. seq.

The individual pluralities of upper 88 and lower 90 cushioning elements are typically constructed of a durable foam or other suitable cushioning and supporting material and are further supported, in individual fashion, in interior facing and extending fashion upon the inner surfaces of each of the upper and lower elongate extending members. In this fashion, the cushioning elements 88 and 90 provide a desired degree of biasing and immobilizing support to the patients upper and lower arm, while still providing air circulation.

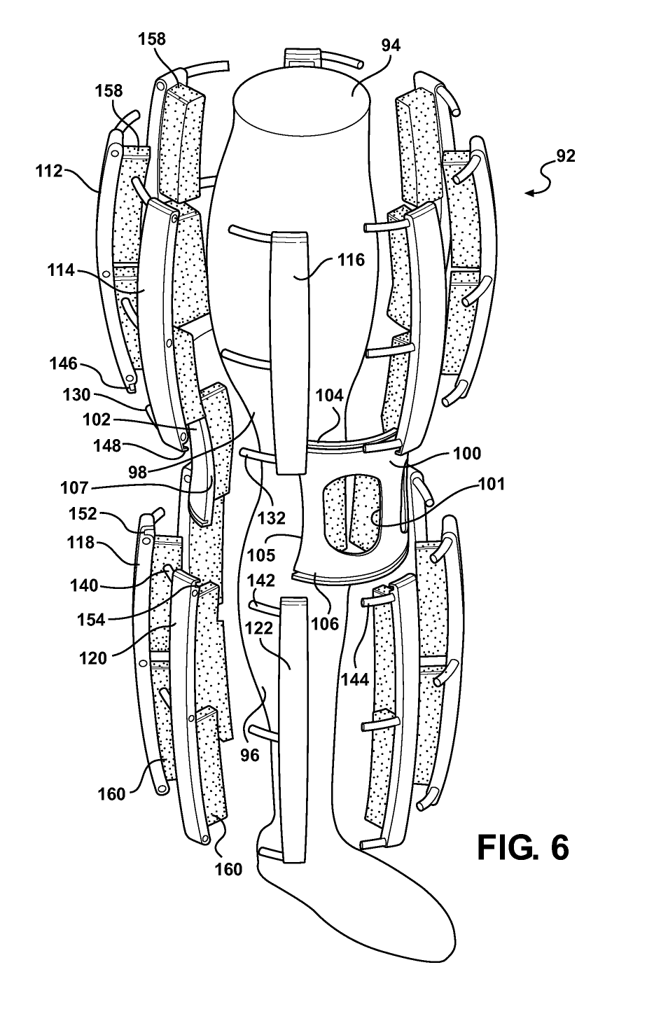

Referring now to FIG. 6, an exploded view is shown at 92 of an adjustable cage splint according to a second preferred embodiment for application to a patient's leg. Similar to the initially disclosed embodiment 10 associated with the patient's arm, the cast assembly 92 discloses another modularized assembly for protecting and immobilizing either or both a patient's upper 94 and lower leg 96, separated by the patient's knee 98.

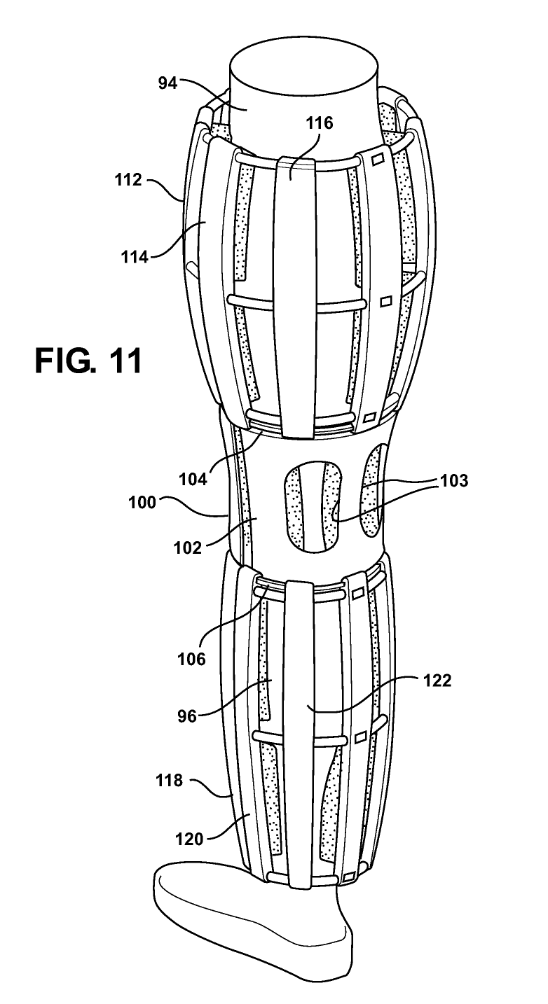

A two piece and assembleable knee joint supporting element (or cast) is shown by components 100 and 102, each of which exhibiting a substantially semi-circular shape with inner and ventilation defined apertures established by inner closed profiles including those shown at 101 for component 100 and at 103 (see FIG. 11) for second assembleable component 102. Opposing end facing locations of the components 100 and 102 included mating and contoured surfaces (see for example as shown at 105 and 107 for components 100 and 102 in FIG. 6) and are capable of being assembled together and so as to define upper 104 and lower 106 rail shaped (this also generally defined as channel or ledge) perimeter extending edges.

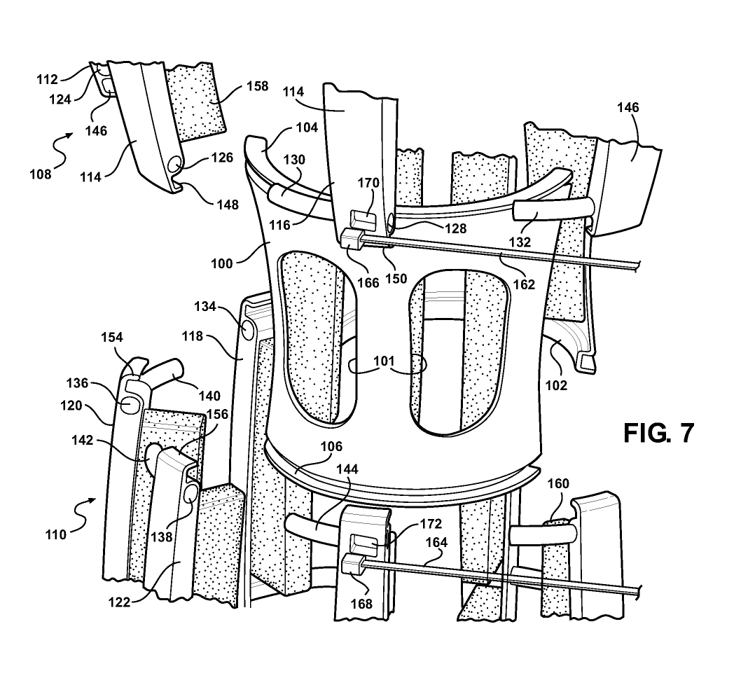

An upper cage subassembly 108 and a lower cage subassembly 110 (see in particular FIG. 7) are provided, each including any plurality of individually assembleable and lengthwise extending sections. The upper subassembly 108 includes, in the illustrated variant, a plurality of such as eight lengthwise elements (see at 112, 114, 116, et. seq.) which assemble together to define a thigh splint, whereas the lower subassembly 110 illustrates a like plurality (e.g. eight) of pieces assembleable (see at 118, 120, 122, et. seq.) to form a calf and lower leg splint.

As best shown in the enlarged exploded view of FIG. 7, better shown are the features associated with the pluralities of assembleable calf and thigh splints engageable with the central, two piece assembleable knee components 100 and 102. Specifically, each of the upper thigh and lower calf lengthwise elements include, at along three lengthwise spaced locations associated with each of the interlocking elements, interlocking features including a receiving aperture located along and within a first side of each element, an extending and interlocking portion extending from an opposite side and seating within an aperture defined in an opposing face of a succeeding element.

As shown in FIG. 7, a lower most ring of apertures 124, 126, 128 et. seq. are formed in upper lengthwise extending (thigh) members 112, 114 and 116, and receive arcuate defined, circumferential projecting and inserting portions (best shown at 130, 132 et seq.), associated with a lower-most mounting location of the multi-piece thigh splint. A corresponding upper most ring of apertures 134, 136, 138, et. seq. are defined such as in the lower lengthwise extending (calf) members 118, 120, 122, these receiving additional arcuate defined, projecting and inserting portions 140, 142, 144, et. seq. associated with associated upper-most and side projection portions extending from succeeding calf members. As best shown in the exploded perspective view of FIG. 6, and without repetitive recitation, each of the upper and lower subassemblies include three tiers of encircling and alternately inter-engaging apertures and circumferential angled and projecting/inserting portions, these in addition to the identified lowermost array associated with upper leg subassembly 108 and uppermost array associated with lower leg subassembly 110, and collectively assembling in order to create an easily assembleable and dimensional adjustable leg cast.

As also shown in FIG. 6, the inter-engaging aspects of the arcuate and perimeter defined apertures and insertion portions are provided at first end, intermediate and second end locations of each of the thigh and calf splint assemblies, this in order to securely assemble each splint about the patient's leg. As again further best shown in each of FIGS. 6 and 7, both the bottom extending edge of the upper thigh splint, as well as the upper extending edge of the lower calf splint, are assembled in engaging fashion about the corresponding upper rail 104 and lower rail 106 of the assembled knee cast.

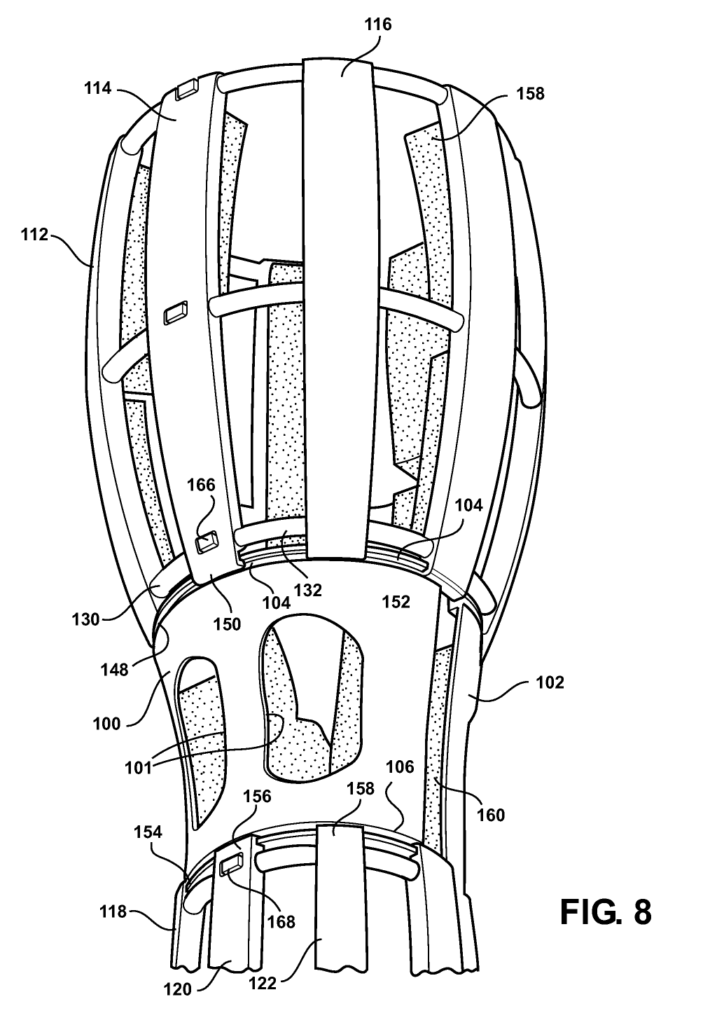

This includes lower projecting edges of the upper thigh splints (see as shown at 146, 148, 150, et. seq. for upper splints 112, 114, 116, et seq.) and upper projecting edges of the lower calf splints (152, 154, 156, et seq.) for lower splints 118, 120 and 122. As shown, each of the lower projecting edges of the splint subassemblies further exhibit arcuate curled and snap-on sections which engages a selected annular location associated with each rail 104 and 106, and as best shown in FIG. 8. As with the arm cast embodiment 10, individual pluralities of foam cushioning elements are shown at 158 and 160, these corresponding to individual (e.g. such as eight apiece) elements associated with both the upper thigh and lower calf splint assemblies 108 and 110.

Diameter adjustment of the thigh and calf splints is provided by encircling bands (tube ties), these corresponding to each spaced apart perimeter interengaging location established for the thigh and calf splints (shown in FIG. 8 by upper/thigh splint exhibiting three spaced apart tie tightening adjustment screws). As best shown in FIG. 7, a pair of encircling bands are shown at 162 and 164, these engaging respectively to lowermost thigh and uppermost calf splint locations approximate the knee joint rails 104 and 106. The bands 162 and 164 terminate in enlarged end locations, see at 166 and 168, which in turn seat and/or are fixedly engaged within surface defined mounting apertures, at 170 and 172 as shown in FIG. 7.

Opposite extending ends of the bands 162 and 164 (not shown) are capable of being engaged within associated end apertures (also not shown) of the bands 162 and 164 and, in one possible variant similar to the tie strip arrangement with serrated engaging edges disclosed in the first embodiment, in order to adjust the diameter of each of the thigh and calf splint assemblies. Other structures can also be employed for diameter adjusting each of the thigh and calf splints, the arrangement of arcuate projecting portions and seating recesses allowing for a reasonable degree of width/diameter adjustment to each splint assembly.

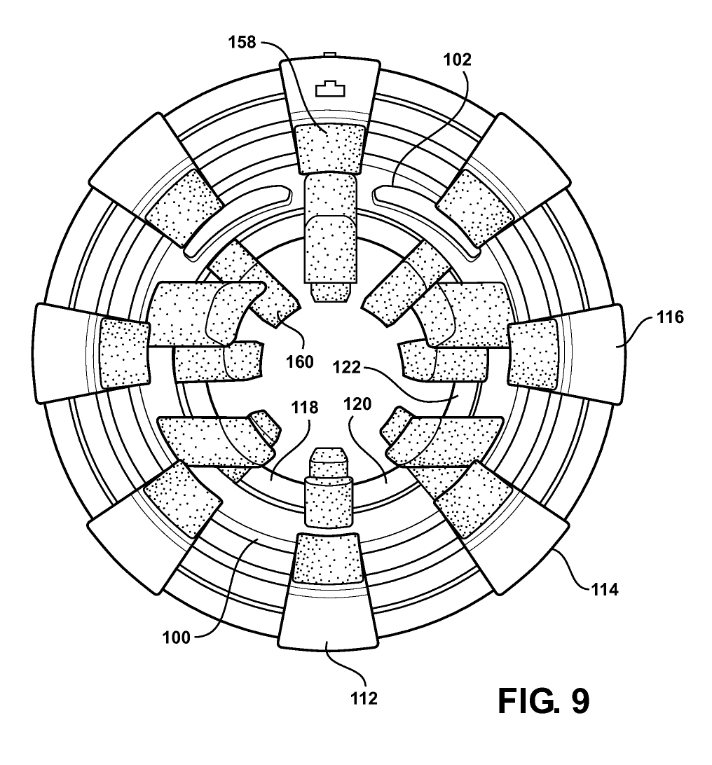

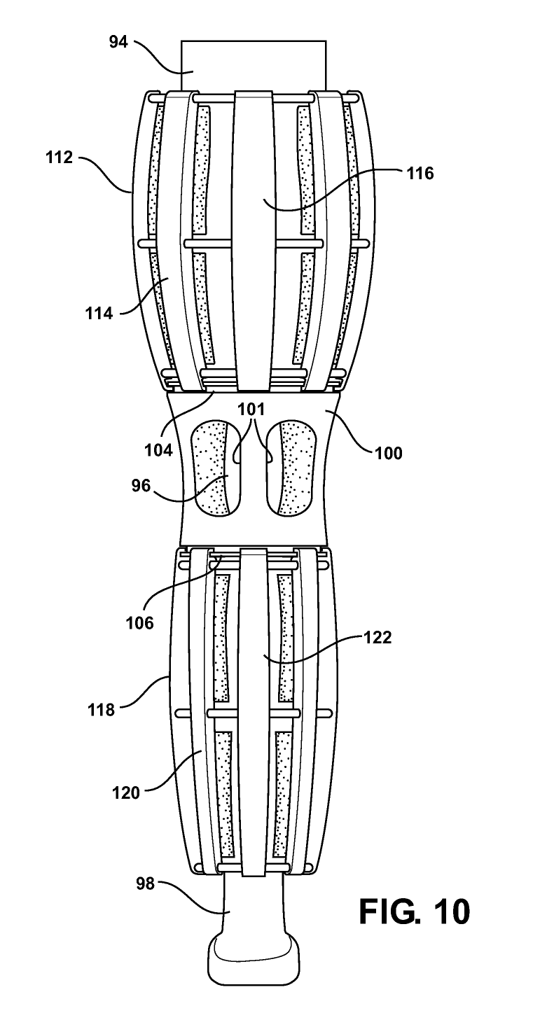

FIG. 8 is an upper assembled perspective view of the leg cast and further illustrating the feature of the tie tightening/adjustment screws 166 and, in similar fashion as previously described in reference to the clamps shown in the embodiment of FIGS. 1-5, the manner in which the upper and lower splint subassemblies align and lock with opposite perimeter extending edges associated with the central assembleable knee portion FIG. 9 is a top view of the leg cast of FIG. 6 and better illustrating the arrangement and configuration of the inner foam supporting portions 158 and 160. FIG. 10 is a front plan view of the assembled leg cast and FIG. 11 is a rotated and rearward looking perspective view of the leg cast.

As with the arm cast of FIGS. 1-5, the leg cast of FIGS. 6-11 provides a modularized assembly which, upon the knee case portion first being assembled over the patient's knee, contemplates either or both the thigh and calf splints be secured to associated rail locations of the knee joint, and in order to selectively immobilize and support the thigh and/or calf of the patient. Having described my invention, other and additional preferred embodiments will become apparent to those skilled in the art to which it pertains, and without deviating from the scope of the appended claims:

I claim:

1. A body limb cast, comprising:
a joint supporting element having a first perimeter edge and a second opposite perimeter edge;
a first splint subassembly secured in engaging and extending fashion from said first perimeter edge, a second splint subassembly secured in engaging and extending fashion from said second perimeter edge of said joint supporting element; and
cushioning elements extending from an inside of each splint subassembly and supporting the surfaces of a patient's limb upon assembly of said joint supporting element and said splint subassemblies.

2. The cast as described in claim 1, said joint supporting element further comprising first and second angled portions corresponding at least one of an elbow joint and a knee joint.

3. The cast as described in claim 1, each of said splint subassemblies further comprising a plurality of individual, lengthwise extending and assembleable members extending around a perimeter about the patient's limb.

4. The cast as described in claim 3, further comprising at least one diameter adjusting clamp associated with each of said first and second splint subassemblies.

5. The cast as described in claim 4, further comprising a band encircling at least one location associated with said splint subassemblies, a diameter of which is adjusted by said adjusting clamp.

6. The cast as described in claim 2, said elbow joint further comprising at least one diameter adjusting clamp.

7. The cast as described in claim 1, said joint supporting element and said splint subassemblies each further comprising a durable and polymeric material.

8. The cast as described in claim 1, said perimeter extending edges of said joint supporting element further comprising a rail support surface upon which opposing locations of said splint subassemblies are resistively engaged.

9. The cast as described in claim 1, said joint supporting element further comprising first and second assembleable halves.

10. The cast as described in claim 4, further comprising a key access feature associated with said diameter adjusting clamp.

11. A body limb cast, comprising:
a joint supporting element having a first perimeter edge and a second opposite perimeter edge;
at least one assembleable splint subassembly further comprising a plurality of individual, lengthwise extending and assembleable members extending around a perimeter about the patient's limb and secured in engaging and extending fashion from a selected one of said first and second perimeter edges of said joint supporting element;
at least one diameter adjusting clamp associated with said at least one splint subassembly, a band encircling at least one location associated with said splint subassembly, a diameter of which is adjusted by said adjusting clamp; and
cushioning elements extending from an inside of each splint subassembly and supporting the surfaces of a patient's limb upon assembly of said joint supporting element and said splint subassembly.

12. The cast as described in claim 11, said joint supporting element further comprising first and second angled portions corresponding at least one of an elbow joint and a knee joint.

13. The cast as described in claim 12, said elbow joint further comprising at least one diameter adjusting clamp.

14. The cast as described in claim 11, said joint supporting element and said splint subassemblies each further comprising a durable and polymeric material.

15. The cast as described in claim 11, said perimeter extending edges of said joint supporting element further comprising a rail support surface upon which opposing locations of said splint subassemblies are resistively engaged.

16. The cast as described in claim 11, said joint supporting element further comprising first and second assembleable halves.

17. The cast as described in claim 11, further comprising a key access feature associated with said diameter adjusting clamp.

18. A body limb cast, comprising:
a joint supporting element exhibiting first and second assembleable portions collectively defining first and second angled sides terminating in a first perimeter edge and a second opposite perimeter edge;
a pair of upper and lower assembleable splint subassemblies further comprising a plurality of individual, lengthwise extending and assembleable members extending around a perimeter about the patient's limb and securing in engaging and extending fashion from a selected one of said first and second perimeter edges of said joint supporting element;
at least one diameter adjusting clamp associated with said at least one splint subassembly, a band encircling at least one location associated with said splint subassembly, a diameter of which is adjusted by said adjusting clamp; and
cushioning elements extending from an inside of each splint subassembly and supporting the surfaces of a patient's limb upon assembly of said joint supporting element and said splint subassembly.
